(19) United States
(12) Patent Application Publication
(10) Pub. No.: US 2012/0010294 A1
(43) Pub. Date: Jan. 12, 2012

(54) COMPOSITIONS AND METHODS FOR THE INHIBITION OF MUSCLE CONTRACTION

(76) Inventor: David A. DILL, Newtown, PA (US)

(21) Appl. No.: 12/956,634

(22) Filed: Nov. 30, 2010

Related U.S. Application Data
(60) Provisional application No. 61/362,374, filed on Jul. 8, 2010.

(51) Int. Cl. A61K 31/192; A61P 13/10

(52) U.S. Cl. 514/570

(57) ABSTRACT

Compositions and methods for the inhibition of muscle contraction are disclosed. In one aspect, the present application discloses pharmaceutical compositions comprising analgesic agents for the inhibition of muscle contraction. In another aspect, the application discloses methods for the prophylactic intervention against premature pain associated with the filling of the urinary bladder of a subject. In yet another aspect, the application discloses methods for the prophylactic intervention against muscle pain associated with physical activity.

This application claims priority from U.S. Provisional Application Ser. No. 61/362,374, filed Jul. 8, 2010. The entirety of that provisional application is incorporated herein by reference.

### FIELD

The present invention generally relates to medical treatment and compositions and, in particular, a method for inhibiting the contraction of muscles.

### BACKGROUND

The detrusor muscle is a layer of the urinary bladder wall made of smooth muscle fibers arranged in spiral, longitudinal, and circular bundles. When the bladder is stretched, this signals the parasympathetic nervous system to contract the detrusor muscle. This encourages the bladder to expel urine through the urethra.

For the urine to exit the bladder, both the autonomically controlled internal sphincter and the voluntarily controlled external sphincter must be opened. Problems with these muscles can lead to incontinence. If the amount of urine reaches 100% of the urinary bladder's absolute capacity, the voluntary sphincter becomes involuntary and the urine will be ejected instantly.

The human adult urinary bladder usually holds about 300-350 ml of urine (the working volume), but a full adult bladder may hold up to about 1000 ml (the absolute volume), varying between individuals. As urine accumulates, the ridges produced by folding of the wall of the bladder (rugae) flatten and the wall of the bladder thins as it stretches, allowing the bladder to store larger amounts of urine without a significant rise in internal pressure.

In most individuals, the desire to urinate usually starts when the volume of urine in the bladder reaches around 125% of its working volume. At this stage it is easy for the subject, if desired, to resist the urge to urinate. As the bladder continues to fill, the desire to urinate becomes stronger and harder to ignore. Eventually, the bladder will fill to the point where the urge to urinate becomes overwhelming, and the subject will no longer be able to ignore it. In some individuals, this desire to urinate starts when the bladder is less than 100% full in relation to its working volume. Such increased desire to urinate may interfere with normal activities, including the ability to sleep for sufficient uninterrupted periods of rest. In some cases, this increased desire to urinate may be associated with medical conditions such as benign prostate hyperplasia or prostate cancer in men, or pregnancy in women. However, increased desire to urinate also occurs in individuals, both male and female, who are not affected by another medical condition.

Accordingly, there exists a need for compositions and methods for the treatment of male and female subjects who suffer from a desire to urinate when the bladder is less than 100% full of urine in relation to its working volume. Said compositions and methods are needed for the inhibition of muscle contraction in order to allow in said subjects the desire to urinate to start when the volume of urine in the bladder exceeds around 100% of its working volume.

### SUMMARY

One aspect of the present invention relates to a method for reducing the frequency of urination. The method comprises administering to a subject in need thereof an effective amount of an analgesic agent.

In a related embodiment, said subject has overactive bladder syndrome.

In another embodiment, said analgesic agent is acetaminophen.

In yet another embodiment, said analgesic agent is a non-steroid anti-inflammatory drug (NSAID). In a preferred embodiment, said NSAID is selected from the group consisting of acetylsalicylic acid, diclofenac, diflunisal, fenoprofen, flurbiprofen, ibuprofen, indomethacin, ketoprofen, ketorolac, meclofenamate, mefenamic acid, naproxen, nambutone, oxaprozin, piroxicam, sulindac, tolmetin, and a coxib.

In a further embodiment, said coxib targets COX-2 more than COX-1. In a preferred embodiment, said coxib is selected from the group consisting of etodolac and meloxicam.

In another further embodiment, said coxib targets COX-2 specifically. In a preferred embodiment, said coxib is celecoxib.

In another embodiment, said analgesic agent is an opiate or morphinomimetic. In a preferred embodiment, said analgesic is codeine or morphine.

In another embodiment, the analgesic agent is formulated for sustained-release or delayed-release.

In another embodiment, the analgesic agent is administered orally.

In another embodiment, the analgesic agent is administered orally as a single dose before bedtime.

In another embodiment, the analgesic agent is administered orally at a dose range of 50-2000 mg.

Another aspect of the present invention relates to a method for prophylactically treating muscle pain or muscle spasms caused by a physical activity. The method comprises administering an effective amount of an analgesic agent prior to the physical activity.

In another embodiment, said analgesic is acetaminophen.

In yet another embodiment, said analgesic is a non-steroid anti-inflammatory drug (NSAID). In a preferred embodiment, said NSAID is selected from the group consisting of acetylsalicylic acid, diclofenac, diflunisal, fenoprofen, flurbiprofen, ibuprofen, indomethacin, ketoprofen, ketorolac, meclofenamate, mefenamic acid, naproxen, nabumetone, oxaprozin, piroxicam, sulindac, tolmetin, and a coxib.

In a further embodiment, said coxib targets COX-2 more than COX-1. In a preferred embodiment, said coxib is selected from the group consisting of etodolac and meloxicam.

In another further embodiment, said coxib targets COX-2 specifically. In a preferred embodiment, said coxib is celecoxib.

In another embodiment, the analgesic agent is formulated for sustained-release or delayed-release.

In another embodiment, the analgesic agent is administered orally.

In another embodiment, the analgesic agent is administered orally as a single dose before a physical activity.

In another embodiment, the analgesic agent is administered orally at a dose range of 50-2000 mg.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

As used herein, the term “effective amount” means an amount necessary to achieve a selected result. Such an amount can be readily determined without undue experimentation by a person of ordinary skill in the art.

As used herein, the term “analgesic” refers to agents, compounds or drugs used to relieve pain and inclusive of anti-inflammatory compounds. Exemplary analgesic and/or anti-inflammatory agents, compounds or drugs include, but are not limited to, the following substances: the salicylates aspirin, salicylic acid, methyl salicylate, diflunisal, salsalate, olsalazine, and sulfasalazine; para-aminophenol derivatives acetanilide, acetaminophen, and phenacetin; the fenamates mefenamic acid, meclofenamate and sodium meclofenamate; the heteroaryl acetic acid derivatives tolmetin, ketorolac and diclofenac; the propionic acid derivatives ibuprofen, naproxen, sodium daproxen, fenoprofen, ketoprofen, flurbiprofen, and oxaprozin; the enolic acids represented by oxicam derivatives piroxicam, meloxicam, tenoxicam, ampiroxicam, droxicam and pivoxicam; the pyrazolon derivatives phenylbutazone, oxyphenbutazone, anitpyrine, aminopyrine and dipyrone; the coxibs celecoxib, and rofecoxib; nabumetone; apazone; nimensulide; indomethacin; sulindac; etodolac; diflunisal, isobutylphenyl propionic acid, and any other substances used in the treatment of pain and inflammatory conditions. As used herein, a “non-aspirin, non-steroidal anti-inflammatory compound” specifically excludes aspirin, acetylsalicylic acid.

As used herein, the terms “coxib” and “COX inhibitor” refer to a composition of compounds that is capable of inhibiting the activity or expression of COX-2 enzymes or is capable of inhibiting or reducing the severity, including pain and swelling, of a severe inflammatory response.

The urinary bladder has two important functions: storage of urine and emptying. Storage of urine occurs at low pressure, which implies that the detrusor muscle relaxes during the filling phase. Emptying of the bladder requires a coordinated contraction of the detrusor muscle and relaxation of the sphincter muscles of the urethra. Disturbances of the storage function may result in lower urinary tract symptoms, such as urgency, frequency, and urge incontinence, the components of the overactive bladder syndrome. The overactive bladder syndrome, which may be due to involuntary contractions of the smooth muscle of the bladder (detrusor) during the storage phase, is a common and underreported problem, the prevalence of which has only recently been assessed.

While not intending to be bound by any particular theory of operation, it is believed that the desire or urge to urinate is caused by pain resulting from the stretching of the bladder muscle and connective tissue as the bladder fills. It is believed that some individuals, both male and female, feel this pain earlier in the filling phase than others, causing them to have an urge that cannot be ignored to urinate before their bladder is 100% full. Accordingly, one aspect of the present invention is to prophylactically provide the subject with an analgesic agent that will inhibit the pain of a filling bladder until the bladder reaches at least 100% full in relation to its working volume and, therefore, reduce the frequency of urination.

In a particular embodiment of the present invention, said analgesic is acetaminophen.

In yet another particular embodiment, said analgesic is a non-steroid anti-inflammatory drug (NSAID). In a preferred embodiment, said NSAID is selected from the group consisting of acetylsalicylic acid (aspirin), diclofenac, diflunisal, fenoprofen, flurbiprofen, ibuprofen, indomethacin, ketoprofen, ketorolac, meclofenamate, mefenamic acid, naproxen, nabumetone, oxaprozin, piroxicam, sulindac, tolmetin, and a coxib.

In a further embodiment, said coxib targets COX-2 more than COX-1. In a preferred embodiment, said coxib is selected from the group consisting of etodolac and meloxicam.

In another further embodiment, said coxib targets COX-2 specifically. In a preferred embodiment, said coxib is celecoxib.

Appropriate dosages of the analgesic agent can be routinely determined by one skilled in the art. In one embodiment, the adult human dose of the analgesic agent is from about 50 mg to about 2000 mg. In another embodiment, the analgesic agent is acetylsalicylic acid and the adult human dose of acetylsalicylic acid is from about 50 mg to about 1000 mg. In a preferred embodiment, the dose of acetylsalicylic acid is from 325 mg to 650 mg. In more preferred embodiments, the dosage of acetylsalicylic acid is 81 mg, 162 mg, 325 mg or 650 mg.

In another embodiment, the analgesic agent is acetaminophen and the adult human dose of acetaminophen is from about 325 mg to about 1300 mg. In a preferred embodiment, the dose of acetaminophen is from 325 mg to 650 mg. In more preferred embodiments, the dosage of acetaminophen is 325 mg, 500 mg, 650 mg or 1300 mg.

In another embodiment, the adult human dose of ibuprofen is from about 100 mg to about 1200 mg. In a preferred embodiment, the dose of ibuprofen is from 400 mg to 800 mg. In more preferred embodiments, the dosage of ibuprofen is 200 mg, 400 mg, or 800 mg.

The present analgesic agents can be formulated into pharmaceutical compositions containing a pharmaceutically acceptable carrier. As used herein, “pharmaceutically acceptable carrier” includes any and all solvents, dispersion media, coatings, antibacterial and antifungal agents, isotonic and absorption delaying agents, sweeteners and the like. The pharmaceutically acceptable carriers may be prepared from a wide range of materials including, but not limited to, flavoring agents, sweetening agents and miscellaneous materials such as buffers and absorbents that may be needed in order to prepare a particular therapeutic composition. The use of such media and agents with pharmaceutically active substances is well known in the art. Except insofar as any conventional media or agent is incompatible with the active ingredient, its use in the therapeutic compositions is contemplated. Optionally, the analgesic agent may be mixed together into pharmaceutical compositions comprising supplementary active ingredients that are not contraindicated by said analgesic agent, such as at least one more analgesic and/or anti-inflammatory substance and/or another compound(s).

In one embodiment, the pharmaceutical composition may be provided in a dry form, and may be formulated into a tablet or capsule form. Tablets may be formulated in accordance with conventional procedures employing solid carriers well-known in the art. Hard and soft capsules employed in the present invention can be made from any pharmaceutically acceptable material, such as gelatin or cellulosic derivatives.

In certain embodiments, the pharmaceutical composition is formulated for sustained-release. Sustained-release, also known as sustained-action (SA), extended-release (ER, XR, or XL), time-release or timed-release, controlled-release (CR), modified release (MR), or continuous-release (CR or Contin), is a mechanism used in medicine tablets or capsules to dissolve slowly and release the active ingredient over time. The advantages of sustained-release tablets or capsules are that they can often be taken less frequently than instant-release formulations of the same drug, and that they keep steadier levels of the drug in the bloodstream, thus extending the duration of the drug action. For example, a sustained-release analgesic may allow a person to sleep through the night without getting up for the bathroom.

In one embodiment, the pharmaceutical composition is formulated for sustained release by embedding the active ingredient in a matrix of insoluble substance(s) such as acrylics or chitin. A sustained release form is designed to release the analgesic compound at a predetermined rate by maintaining a constant drug level for a specific period of time. This can be achieved through a variety of formulations, including, but not limited to liposomes and drug-polymer conjugates, such as hydrogels.

In another embodiment, the pharmaceutical composition is formulated for delayed-release, such that the active ingredient(s) is not immediately released upon administration. A non-limiting example of a delayed release vehicle is an enteric coated oral medication that dissolves in the intestines rather than the stomach. In an optimal scenario for someone wishing to limit interference with restful sleep, the vehicle would release the medicine at the point when the individual would normally be awakened by an urge to urinate. For example, consider an individual who begins sleeping at 11 PM and is normally awakened at 12:30 AM, 3:00 AM, and 6:00 AM to urinate. A delayed release vehicle could deliver the medicine at 12:15 AM, thereby delaying the need to urinate for perhaps 2-3 hours. After finally arising to urinate at now 3 AM for the first time, typically the individual may not be again awakened until 7 AM or later. Eliminating one or more trips to the bathroom during the night may be the most common use of this embodiment.

In a preferred embodiment, the analgesic compound is delivered in a vehicle that is both delayed release and sustained release.

In other embodiments, the pharmaceutical composition is formulated for rapid release of the analgesic agent. In one embodiment, the pharmaceutical composition is formulated as a powder that can be ingested for rapid release of the analgesic agent. In another embodiment, the pharmaceutical composition is formulated into a liquid, gel or emulsion form. Said liquid, gel or emulsion may be ingested by the subject in naked form or contained within a capsule.

In yet another embodiment, the pharmaceutical composition may be provided as a skin or transdermal patch for the topical administration of controlled and/or sustained quantities of the analgesic agent.

The pharmaceutical composition may be administered daily or administered on an as needed basis. In certain embodiments, the pharmaceutical composition is administered to the subject prior to bedtime. In some embodiments, the pharmaceutical composition is administered immediately before bedtime. In some embodiments, the pharmaceutical composition is administered within about two hours before bedtime, preferably within about one hour before bedtime. In another embodiment, the pharmaceutical composition is administered about two hours before bedtime. In a further embodiment, the pharmaceutical composition is administered at least two hours before bedtime. In another embodiment, the pharmaceutical composition is administered about one hour before bedtime. In a further embodiment, the pharmaceutical composition is administered at least one hour before bedtime. In a still further embodiment, the pharmaceutical composition is administered less than one hour before bedtime. In still another embodiment, the pharmaceutical composition is administered immediately before bedtime. Preferably, the pharmaceutical composition is administered orally. Suitable compositions for oral administration include, but are not limited to: tablets, coated tablets, dragees, capsules, powders, granulates and soluble tablets, and liquid forms, for example, suspensions, dispersions or solutions.

In another particular embodiment, the analgesic compound is administered in an effective amount to the subject by rectal administration as a suppository.

Another aspect of the present invention relates to a method for prophylactically treating muscle pain or muscle spasms caused by a physical activity. The method comprises administering an effective amount of an analgesic agent prior to the physical activity. In some embodiments, the pharmaceutical composition is administered within about two hours before the physical activity, preferably within about one hour before the physical activity. In another embodiment, the pharmaceutical composition is administered about two hours before the physical activity. In a further embodiment, the pharmaceutical composition is administered at least two hours before the physical activity. In another embodiment, the pharmaceutical composition is administered about two to four hours before the physical activity. In a further embodiment, the pharmaceutical composition is administered less than one hour before the physical activity. In still another embodiment, the pharmaceutical composition is administered immediately before the physical activity. Preferably, the pharmaceutical composition is administered orally. Suitable compositions for oral administration include, but are not limited to: tablets, coated tablets, dragees, capsules, powders, granulates and soluble tablets, and liquid forms, for example, suspensions, dispersions or solutions.

In another embodiment, said analgesic is acetaminophen.

In yet another embodiment, said analgesic is a non-steroid anti-inflammatory drug (NSAID). In a preferred embodiment, said NSAID is selected from the group consisting of acetylsalicylic acid, diclofenac, diflunisal, fenoprofen, flurbiprofen, ibuprofen, indomethacin, ketoprofen, ketorolac, meclofenamate, mefenamic acid, naproxen, nabumetone, oxaprozin, piroxicam, sulindac, tolmetin, and a coxib.

In a further embodiment, said coxib targets COX-2 more than COX-1. In a preferred embodiment, said coxib is selected from the group consisting of etodolac and meloxicam.

In another further embodiment, said coxib targets COX-2 specifically. In a preferred embodiment, said coxib is celecoxib.

In another embodiment, the analgesic agent is formulated for sustained-release or delayed-release. For example, if a person who habitually ended up feeling pain after an extended period of certain activity, such person may take a sustained-release pill in advance to cover the period of activity. This would especially be true if it were not convenient to take a pill in the midst of that activity. A delayed-release embodiment may also be useful for overnight use since one's urge to urinate may not need to be modified for the first hour or two of sleeping time.

In another embodiment, the analgesic agent is administered orally.

In another embodiment, the analgesic agent is administered orally as a single dose before a physical activity.

In another embodiment, the analgesic agent is administered orally at a dose range of 50-2000 mg.

The present invention is further illustrated by the following example which should not be construed as limiting. The contents of all references, patents and published patent applications cited throughout this application are incorporated herein by reference.

### EXAMPLE 1

### Inhibition of the Urge to Urinate

Twenty volunteer subjects, both male and female were enrolled, each of which experienced premature urge or desire to urinate, interfering with their ability to sleep for a sufficient period of time to feel adequately rested. Each subject ingested 400-800 mg of ibuprofen as a single dose prior to bedtime. At least 14 subjects reported that they were able to rest better because they were not being awakened as frequently by the urge to urinate.

Several subjects reported that after several weeks of nightly use of ibuprofen, the benefit of less frequent urges to urinate was no longer being realized. However, all of these subjects further reported the return of the benefit after several days of abstaining from taking the dosages.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

What is claimed is:

1. A method for reducing the frequency of urination, comprising:
administering to a subject in need thereof an effective amount of an analgesic agent.

2. The method of claim 1, wherein said subject has overactive bladder syndrome.

3. The method of claim 1, wherein said analgesic agent is acetaminophen.

4. The method of claim 1, wherein said analgesic agent is a non-steroid anti-inflammatory drug (NSAID).

5. The method of claim 4, wherein said NSAID is selected from the group consisting of acetylsalicylic acid, diclofenac, diflunisal, fenoprofen, flurbiprofen, ibuprofen, indomethacin, ketoprofen, ketorolac, meclofenamate, mefenamic acid, naproxen, nabumetone, oxaprozin, piroxicam, sulindac, tolmetin, and a coxib.

6. The method of claim 1, wherein said analgesic agent is formulated for sustained-release or delayed-release.

7. The method of claim 1, wherein said analgesic agent is formulated for both sustained-release and delayed-release.

8. The method of claim 1, wherein said analgesic agent is administered orally.

9. The method of claim 1, wherein said analgesic agent is administered as a single dose before bedtime.

10. The method of claim 1, wherein said analgesic agent is administered at a dose range of 50-2000 mg.

11. The method of claim 1, wherein said analgesic is administered at a time selected from the group consisting of: at bedtime, less than one hour before bedtime, at least one hour before bedtime, one to two hours before bedtime and at least two hours before bedtime.

12. A method for prophylactically treating muscle pain or muscle spasms caused by a physical activity comprising:
administering an effective amount of an analgesic agent prior to the physical activity.

13. The method of claim 12, wherein said analgesic is acetaminophen.

14. The method of claim 12, wherein said analgesic is a non-steroid anti-inflammatory drug (NSAID).

15. The method of claim 14, wherein said NSAID is selected from the group consisting of acetylsalicylic acid, diclofenac, diflunisal, fenoprofen, fluriprofen, ibuprofen, indomethacin, ketoprofen, ketorolac, meclofenamate, mefenamic acid, naproxen, nabumetone, oxaprozin, piroxicam, sulindac, tolmetin, and a coxib.

16. The method of claim 12, wherein said analgesic agent is formulated for sustained-release or delayed-release.

17. The method of claim 12, wherein said analgesic agent is formulated for both sustained-release and delayed-release.

18. The method of claim 12, wherein said analgesic agent is administered orally.

19. The method of claim 12, wherein said analgesic agent is administered as a single dose before a physical activity.

20. The method of claim 12, wherein said analgesic agent is administered at a dose range of 50-2000 mg.
